(19) United States
(12) Patent Application Publication
(10) Pub. No.: US 2009/0130181 A1
(43) Pub. Date: May 21, 2009

(54) Antibacterial Surface Treatments Based on Silver Cluster Deposition

(76) Inventors: Mauro Pollini, Lizzanello (Lecce) (IT); Alessandro Sannino, Lecce (IT); Alfonso Maffezzoli, Lecce (IT); Antonio Licciulli, Mesagne(Brindisi) (IT)

Correspondence Address: R. Ruschena Patent Agent, LLC, 8400 E. Crescent Parkway, Suite 600, Greenwood Village, CO, 80111, US

(21) Appl. No.: 12/087,326

(22) PCT Filed: Dec. 28, 2005
(86) PCT No.: PCT/IT2005/000772

§ 371 Date: Jun. 27, 2008

(51) Int. Cl. A61K 9/70; C08J 7/18; A61P 31/04

(52) U.S. Cl. 424/443; 427/553

(57) ABSTRACT

Process to obtain antibacterial surfaces by silver deposition in the form of firmly bonded small particles and to the antibacterial substances obtained by aforementioned treatments. Silver deposition is obtained by surface impregnation of natural or synthetic material in an alcoholic solution with silver salt and, later, by their exposure to UV-rays until metal silver clusters form as a result of silver ions reduction on the material surface. The invention relates to the obtained antibacterial substances.

The simple preparation of the antibacterial material makes the whole process easier both for required time and for costs: the needed devices are just a UV lamp and an Ultrasound bath.

### TECHNICAL FIELD

The present invention relates to a process to obtain antibacterial surfaces by silver deposition in the form of firmly bonded small particles and to the antibacterial substances obtained by aforementioned treatments.

Silver has been known as a purifying agent since the Egyptian age when it was employed to purify water to be stored for a long period of time. Modern medicine makes use of silver as an antibacterial agent in the treatment of burns or eye infections in newborn babies, see M. Potenza, G. Levinsons, AIM 59 (2004). Since the last century silver solutions have been used as an antibacterial agent to help cure infected wounds, and is used for the water purification system on the NASA space shuttle. The anti-inflammatory properties of silver have been proved by a reduced reddening of infected wounds edges. Other heavy metal, such as zinc, lead, gold, nickel, cadmium, copper and mercury are also known to have anti-bacterial properties, but some of them cannot be not used because of their toxicity or because of high costs. Among heavy metals, only silver, zinc and copper can be used as antibacterial agents. Zinc is less effective than the others; while copper, though highly effective against some mildews, when combined with silver has a synergic effect, however it cannot be used in contact with food. Silver ion is the most effective ion with the lowest toxicity. On this subject, see: J. M. Schierholz, L. J. Lucas, A. Rump, G. Pulverer, Journal of Hospital Infection (1008) 40: 257-262; Gadd G M, Laurence O S, Briscoe P A, Trevors J T. Silver accumulation in Pseudomonas stutzeri AG 259. Bio Metals 1989; 2: 168-173; Wahlberg J E. Percutaneous toxicity of metal compounds. Arch Environ Health 1989; 11: 201-203; Williams R L, Williams D F. Albumin adsorption on metal surfaces. Biomat 1988; 9: 206.

The material releases silver ions that attach themselves to the bacteria, incapacitating them and preventing them from growing or reproducing. Therefore, a silver-based antibacterial product cannot be everlasting, because its silver quantity will decrease in time. When released by the material, silver ions act on the bacteria (see Y. Noue, Y. Kanzaki, Enviromental Bioinorganic Chemistry, Journal of inorganic Biochemistry 377), according to a still unknown mechanism, which can be summarized in this way: when silver is ingested by the bacterium, it destroys its cell walls, inhibits its reproduction and stops its metabolism, see M. Potenza, G. Levinsons, AIM 59 (2004). Silver has no toxic effect on living human cells. It has a very powerful antibacterial property, since a solution with only 1 ppm of pure elemental silver has an effective bacterial killing action. Natural or synthetic materials (e.g. fabric, woven and similar), with antibacterial properties have already been realized in several fields, such as clothing, medicine, filtering systems, transportation and others. They have different shapes and trade-names but all of them are very expensive, because of the existing difficulties in their realization.

Blowes and Tayloe (see WO/49219 A (Foxwood res ltd [GB] Blowes Phillip Charles [GB] Tayloe Alan John [GB]) 24 Aug. 2000) use chitosan coating, impregnated with a silver salt solution. Silver is not film former nor reduced in cluster form.

Yuranova et al (see Yuranova et Al. “Antibacterial textiles prepared by RF-plasma and vacuum-UV mediated deposition of silver”, Journal of Photochemistry and photobiology, A: Chemistry, 161 (1), 27-34.2) used UV light for the chemical activation of textile substrates subsequently impregnated in a silver salt solution. The reduction is obtained on such activated textile through the chemical reduction of a silver salt.

Gaddy et al. (see Gaddy, G. A. et Al.: “Photogeneration of silver particles in PVA fibers and films”, Journal of cluster science, 12 (3), 457-471) produced PVA film. Silver particles are nucleated inside the PVA matrix. The photoreduction of silver ions in to silver cluster occurs in the PVA matrix. PVA acts as a reducing agent for the metal ions in the presence of UV light.

Hada et al. (see Hada, Hiroshi et Al.: “Photoreduction of silver ion in aqueous and alcoholic solutions” Journal of physical chemistry” 80 (25)) report photoreduction of a silver perchlorate salt in solution. Photoreduction of silver in solution is not effective to form a stable well adhered silver coating. Similarly Yan Jixiong et al. (se WO 03/080911 A2 (CC technology Invest Co LTD [CN]; Yan Jixiong [CN] Soh Kar Liang [SG]) 2 Oct. 2003) report a nanoparticle silver coating obtained depositing silver nanoparticles previously reduced in solution through chemical reducing agents: The nanosilver particle-containing solution was prepared by mixing the silver nitrate solution with the reducing agent solution.

### DISCLOSURE OF THE INVENTION

The invention relates to a process for antibacterial treatments characterized by its simplicity and inventiveness due to the fact that it uses no binders, additional materials and complex reducing agents. Natural or synthetic materials are impregnated with alcohol solution of silver salt and methanol, eventually dilute in water or other alcoholic solvents. In the second step of the process, the dried substances are exposed to UV-rays until the metal silver clusters form on the material surface. The invention also relates to the obtained antibacterial substances.

The simplicity of the antibacterial material preparation makes the whole process easier both for required time and for costs: the equipment needed to carry on the procedure comprises a UV lamp and an ultrasounds bath.

These and other advantages will be pointed out in the detailed description of the invention that will refer to the figures of the tables 1/3, 2/3 and 3/3, each of them exemplifying and not restrictive.

### WAY OF CARRYING OUT THE INVENTION

With reference to the above mentioned tables:

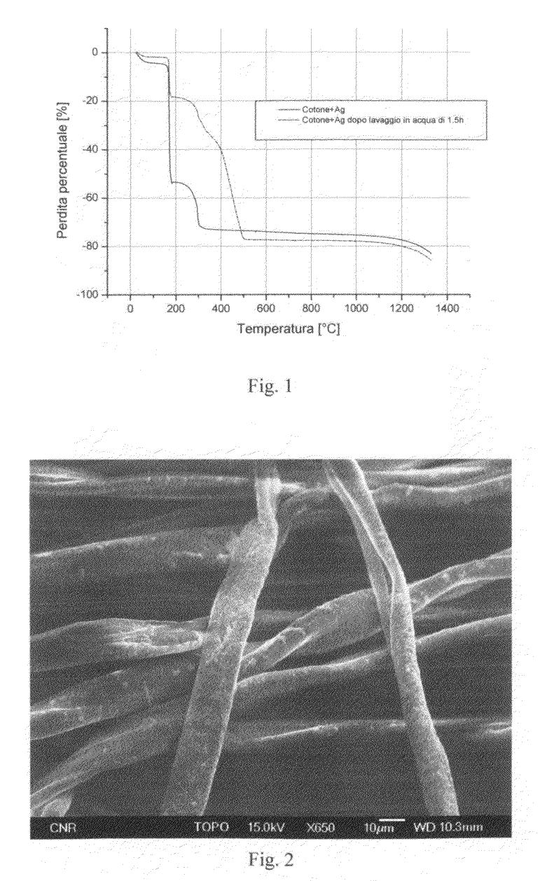

FIG. 1 shows the results of a thermal-gravimetric analysis;

FIG. 2 is a S.E.M. representation (650×) of the 100% cotton fibers, impregnated with the silver;

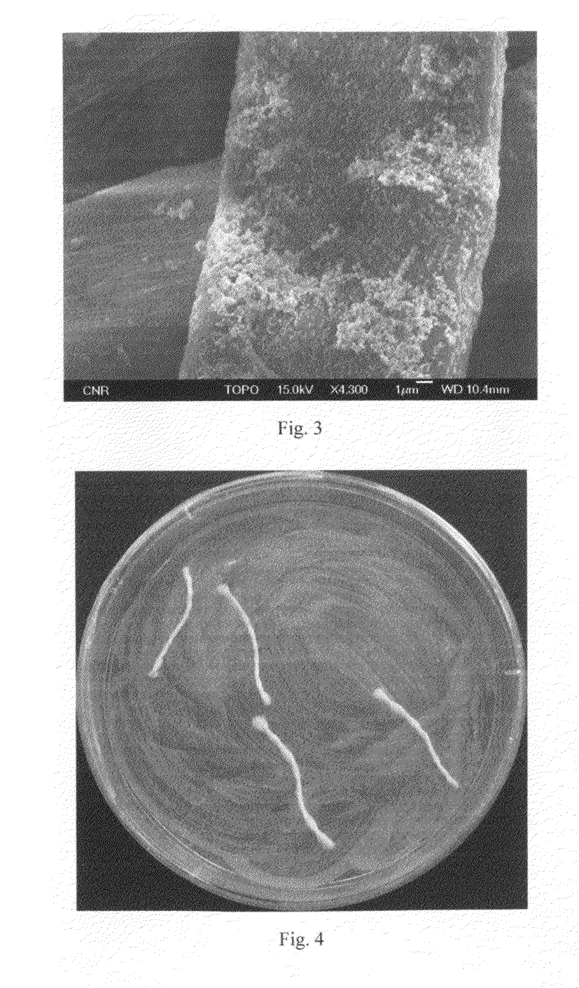

FIG. 3 is a S.E.M. representation (4300×) of the 100% cotton fibers, impregnated with silver;

FIG. 4 shows a growth test of Escherichia coli JM101 AMERSHAM on a 100% cotton sample;

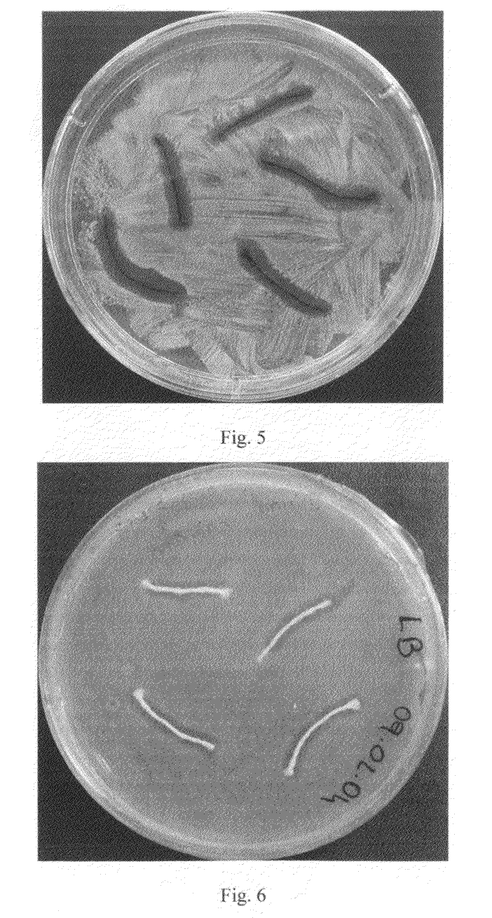

FIG. 5 shows a growth test of Escherichia coli JM101 AMERSHAM on a cotton sample, impregnated with silver;

FIG. 6 shows a growth test of Escherichia coli JM101 AMERSHAM on a cotton sample, impregnated with kanamicina antibiotic.

The first step of the procedure is the preparation of the silver solution; containing a silver salt (for example, silver nitrate AgNO₃) in alcohol solvent (for example, methanol). Other silver salts, such as silver chloride or silver acetate, can be used as well. The weight ratio of the solvent to the solute is strictly dependent on the silver quantity to be deposited on the material. A typical example is a solution at 5%_{wt} of silver nitrate in methanol. The best dissolution of silver salt in alcohol can be achieved when the solution is exposed to ultrasound rays for few minutes, until completely homogenous. Moreover, according to laboratory tests, the solution can be stored for a long time in dark keeping conditions, without loosing its effectiveness. This helps the industrialization process, because the solution would be ready to be used whenever a material needs to be impregnated. However, the long-stored solution should be newly exposed to the ultrasound bath, when the weight percentage of the silver in the solution is high (like 5%).

A minimum methanol content is prescribed since methanol is the activator of the silver UV reduction. Such content is given with respect to Ag content in the solution, lower methanol content is not effective for reducing silver ions. The methanol is responsible for the formation of silver nanoparticles through the formation of a methanol radical.

Although, the methanol radical alone does not possess the required negative potential to reduce the silver ions but it needs photons absorption.

An advantage of the present invention is that silver ions reduction takes place with UV irradiation only after the solution has been applied on the surface. The silver particles will consequently form with a strong adhesion to the substrate. The reduced dimension and the enhanced surface area of the small particles will provide a very high antibacterial activity.

As a consequence of the above description of the physical and chemical mechanisms of coating formation, the silver impregnation protocol of fibre, woven or, in general, the material, is according to the 3 following steps:
1. Apply the solution to the material through any of the well-known wet coating methods: dip coating, spray coating, laminar coating, spin coating.
2. Expose the wet material to UV rays.
3. Dry the material.

The end silver clusters are strongly bonded to the substrate.

As a result, the material changes its colour, for example from white to dark brown. To form and fix the metal silver clusters to the material a radiation power range between 20 W/m² and 10000 W/m² is needed with an exposure time between 5 sec and 30 minutes, and a wavelength between 285 and 400 nm. In a preferred procedure, the distance of the lamp from the sample surface is 10 cm, corresponding to a power of 500 W/m² and an exposure time between 1 and 2 minutes. In FIG. 1 the results of a thermal-gravimetric analysis have been shown for a comparison between a not washed 100% cotton sample and a washed (1,5 h) 100% cotton sample. On both, silver has been deposited, starting from a 5%_{wt} silver nitrate solution. TGA curves show a fix residual equal to 21.51%_{wt} in the first case and 18.74%_{wt} in second case (washed fibre). These percentages do not include the cotton fix residual, which is 3.6%. In FIGS. 2 and 3, images from the scan electronic microscope (SEM) are shown: they are related to 100% cotton fibres on which silver has been deposited. Above all in FIG. 3 (4300×), the metallic clusters are perfectly visible.

The antibacterial effectiveness has been checked (but not exclusively) with Escherichia coli JM101 AMERSHAM bacterial cultivation. The test has been carried out on several samples, even those treated with antibiotic. The testing slabs have been previously filled by agar, which is an excellent medium for growing soil bacteria. Once the agar became solid, 1 ml bacterial suspension has been injected into each slab and distributed on the whole agar surface. Then the fibre samples have been introduced and the slabs have been put in an oven at 37° C. for 24 h. The results in FIGS. 4-6 show a remarkable antibacterial property of the fibre, which were treated according to the present invention: their performance is equal or even better than the one of fibres, which were impregnated with the kanamicina antibiotic. The bacterial growth inhibition areas have to be evaluated by measuring the area surrounding the sample, in which no bacterial proliferation can be seen. From FIG. 4, you can observe that the 100% cotton does not show any antibacterial behaviour. In FIG. 5, which is related to fibres impregnated with silver, according to the present invention, the antibacterial behaviour is shown: a well-defined area around the sample, without bacterial proliferation, can be noted. Same antibacterial behaviour is shown in FIG. 6, which is related to a cotton sample, impregnated with kanamicina antibiotic. A slight different process consists in the fact that the deposit of the silver solution on the material can be realized by spraying the solution by an airbrush. In the following step, the exposure to the UV rays, does not change.

### EXAMPLE 1

An example of carrying out the process is the described below.

a) Solution Preparation

For 100 g solution with 5%_{wt} of silver nitrate, you would need 95.24 g methanol and 4.76 g AgNO₃.

Dilute the silver salt in the methanol, by dipping the beaker in an ultrasounds bath for five minutes.

b) Impregnation

Shortly dip the fibers inside the beaker containing the solution; then expose them to the UV rays for approximately two minutes; the silver clusters will appear together with a color change in the fibers, which, if white, will become dark brown.

### EXAMPLE 2

Another example of carrying out the process is described below.

c) Solution Preparation

For 100 g aqueous solution with 5%_{wt} of silver nitrate, you would need 10 g methanol and 5 g AgNO₃ and 85 g H₂O.

Dilute the silver salt in the methanol, than add water.

d) Impregnation

Shortly dip the fibers inside the beaker, containing the solution; then expose them to the UV rays for approximately two minutes; a color change in the fibers, will indicate that silver ions reduction to form silver clusters has occurred.

What we claim is:

1) A process for obtaining an antibacterial coatings by impregnation of natural or synthetic materials in a solution comprising alcohol and silver salt, characterized in that:
said alcohol is methanol, acting as reducing agent;
afterward, the impregnated substrate is exposed to UV-rays until the metal silver clusters are formed and bonded on the material surface.

2) Process according to claim 1, wherein said materials are natural or synthetic fibers; said fibers maybe present in the form of single yarn, fabric, or non-woven fiber.

3) Process according to claim 1, wherein said salt is silver nitrate and note the weight percentage of the silver in the solution (like 5%), the quantity of methanol is not inferior to a minimum amount able to reduce silver ions.

4) Process according to claim 1, wherein said exposure to UV-rays is characterized by the following ranges: wave length 285÷400 nm, power 20÷10000 W/m2 and exposure time between 5 seconds and 30 minutes.

5) Antibacterial fibers characterized by the fact that they are obtainable by surface impregnation in a solution containing methanol and silver nitrate and, later, by exposing said natural or synthetic material to UV-rays until metal silver clusters form and cohere to fibers.

6) Antibacterial natural fabric according to claim 5, wherein said fabric is made of cotton.

7) Antibacterial natural fabric according to claim 5, wherein said exposure to UV-rays is characterized by the following ranges: wave length 285÷400 nm, power 20÷10000 W/m2 and exposure time between 5 seconds and 30 minutes.

8) Antibacterial polymeric fabric according to claim 5, wherein said exposure to UV-rays is characterized by the following ranges: wave length 285÷400 nm, power 20÷10000 W/m2 and exposure time between 5 seconds and 30 minutes.

9) Antibacterial natural woven according to claim 5, wherein said exposure to UV-rays is characterized by the following ranges: wave length 285÷400 nm, power 20≦10000 W/m2 and exposure time between 5 seconds and 30 minutes.

10) Antibacterial polymeric woven according to claim 5, wherein said exposure to UV-rays is characterized by the following ranges: wave length 285÷400 nm, power 20÷10000 W/m2 and exposure time between 5 seconds and 30 minutes.

11) Antibacterial woven/non woven fabric according to claim 5, wherein said exposure to UV-rays is characterized by the following ranges: wave length 285÷400 nm, power 20÷10000 W/m2 and exposure time between 5 seconds and 30 minutes.
